(19) United States
(12) Patent Application Publication
(10) Pub. No.: US 2008/0132988 A1
(43) Pub. Date: Jun. 5, 2008

(54) Balloon geometry for delivery and deployment of shape memory polymer stent with flares

(75) Inventor: Gary Jordan, Litchfield, NH (US)

Correspondence Address: HOFFMANN & BARON, LLP, 6900 JERICHO TURNPIKE, SYOSSET, NY, 11791, US

(73) Assignee: SCIMED Life Systems, Inc.

(21) Appl. No.: 11/607,611

(22) Filed: Dec. 1, 2006

(51) Int. Cl. A61F 2/06

(52) U.S. Cl. 623/1.11

(57) ABSTRACT

A balloon geometry is utilized herein where the balloon is inflatable from an initial unexpanded state to an expanded state. The balloon includes first and second portions; the first portion having a first diameter with the balloon being in the expanded state, the second portion having a second diameter with the balloon being in the expanded state, the first diameter being different from the second diameter. With a stent being mounted onto the balloon, expansion of the balloon results in the first portion of the balloon assisting a first portion of the stent to radially expand more than a second portion of the stent located adjacent to the second portion of the balloon. Preferably, the stent is formed of shape memory polymer. With the subject invention, one or both ends of the stent can be formed with larger diameters, or flares, in vivo at the point of implantation. The flared ends provide engagement points for the stent to a surrounding bodily passageway. Although the balloon may have various applications, it is particularly well-suited for use with shape memory polymer stents, which can be expanded and deformed in vivo.

### FIELD OF THE INVENTION

This invention relates to balloons for radially expanding stents, and more particularly, to balloons for radially expanding shape memory polymer (SMP) stents.

### BACKGROUND OF THE INVENTION

Shape memory polymer (SMP) stents are known in the prior art. The following disclosures, which are all incorporated by reference herein, disclose suitable materials and geometries for SMP stents: U.S. Published Patent Appl. No. 2005/0010275; PCT Published Patent Appl. No. WO 2004/032799; U.S. Published Patent Appl. No. 2005/0216074; U.S. Published Patent Appl. No. 2005/0251249; U.S. Published Patent Appl. No. 2005/0245719; U.S. Published Patent Appl. No. 2004/0116641; PCT Published Patent Appl. No. WO 2004/033515; U.S. Published Patent Appl. No. 2004/0122174; PCT Published Patent Appl. No. WO 2004/033539; U.S. Published Patent Appl. No. 2004/0083439; PCT Published Patent Appl. No. WO 2004/033553; U.S. Published Patent Appl. No. 2005/0075625; and, PCT Published Patent Appl. No. WO 2005/009523.

Stents are often used in the gastrointestinal tract to treat malignant or benign strictures as palliative or supporting treatment to chemotherapy or surgery. With biliary stent applications, plastic stents are often used. Plastic stents are typically 2-3 mm in diameter and need to be exchanged relatively often (e.g., every three months) due to occlusion from bile. Metal stents, such as self-expanding metal stents, are also useable and tend to have a longer patency than plastic stents because of their larger diameters, typically 8-10 mm. However, plastic stents may be removable, whereas, metal stents generally are not. Common practice calls for removing stents when treatment of benign strictures is completed. Accordingly, metal stents are generally restricted to use where malignant, not benign, strictures are present.

A need had developed in the prior art for a stent having relatively large diameters in the range of metal stents, e.g., 8-10 mm, yet, be removable. SMP stents satisfy this need with SMP stents both being useable at the relatively large diameters, thereby providing good patency, and being removable, thus allowing for use with both benign and malignant applications.

SMP stents are formable as tubular structures (which may be cut or etched or otherwise have material removed) or as coiled structures resembling coil springs. With either configuration, a straight, generally cylindrical shape may not be desired, due to the possibility of migration within a bodily passageway. A method has been developed of pre-forming SMP stents with one or both ends flared, with the SMP stents recovering this configuration in vivo at the point of implantation. However, in preparing the SMP stents, the stents are initially pre-formed with the flared-end configuration and then contracted to a minimized diameter for insertion into a catheter (in being readied for implantation) and later expanded. Alternatively, the SMP stents may be formed at a reduced profile and expanded to a desired size. The pre-expansion profile of the SMP stents resembles proportionately the profile of the fully-expanded stents, with the ends being likewise flared. With the smallest possible profile being sought for insertion into a patient, the flared-end configurations of the contracted SMP stents may be undesirable.

### SUMMARY OF THE INVENTION

A balloon geometry is utilized herein where the balloon is inflatable from an initial unexpanded state to an expanded state. The balloon includes first and second portions; the first portion having a first diameter with the balloon being in the expanded state, the second portion having a second diameter with the balloon being in the expanded state, the first diameter being different from the second diameter. With a stent being mounted onto the balloon, expansion of the balloon results in the first portion of the balloon assisting a first portion of the stent to radially expand more than a second portion of the stent located adjacent to the second portion of the balloon. Preferably, the stent is formed of SMP. With the subject invention, one or both ends of the stent can be formed with larger diameters, or flares, in vivo at the point of implantation. The flared ends provide engagement points for the stent to a surrounding bodily passageway. Although the balloon may have various applications, it is particularly well-suited for use with SMP stents, which can be expanded and deformed in vivo.

The balloon is catheter mounted and useable in various bodily passageways for implanting a stent, including, but not limited to, the gastrointestinal tract (e.g, bile ducts, colon, duodenum), esophagus, bronchi, trachea, urine tract (e.g., urethra, ureter, prostate) and vasculature (e.g., coronary blood vessels, peripheral blood vessels, intracranial blood vessels).

These and other features will be better understood through a study of the following detailed description and accompanying drawings.

### BRIEF DESCRIPTION OF THE DRAWINGS

FIG. 1 is a schematic of an assembly including a catheter, with a balloon in an unexpanded state being located thereon, and a stent mounted onto the balloon in accordance with one embodiment of the invention;

FIG. 2 is a schematic of a balloon in an expanded state in accordance with one embodiment of the invention;

FIGS. 3(a)-(e) are schematics of various alternative balloon geometries in accordance with alternate embodiments of the invention;

FIG. 4 is a schematic of an expanded balloon located in a bodily passageway having a tubular stent thereabout in accordance with one embodiment of the invention:

FIG. 5 is a schematic of an expanded balloon having a coiled stent thereabout in accordance with one embodiment of the invention; and,

FIGS. 6(a)-(c) are schematics of alternative balloon configurations in accordance with alternate embodiments of the invention.

### DETAILED DESCRIPTION OF THE INVENTION

A balloon 10 is provided herein for expanding and/or deforming a stent 12 in vivo at the point of implantation. The balloon 10 is located on a catheter 14, as is known in the art. The catheter 14 may be formed in accordance with any known design and includes a proximal end 16 and a distal end 18. With the distal end 18 being intended for insertion into a patient, it is preferred that the balloon 10 be located in proximity to the distal end 18.

With reference to FIG. 2, the balloon 10 may extend along a longitudinal length of the catheter 14 and includes first and second ends 20 and 22, respectively, and an intermediate portion 24 therebetween. The balloon 10 is expandable from an initial unexpanded state to an expanded state.

With the subject invention, at least one portion of the balloon 10 is formed to expand to a different, e.g. larger, diameter than one or more other portions of the balloon 10. For example, as shown in FIG. 2, a first portion 26, located in proximity to the first end 20, is expandable to a diameter D1. A second portion 28 of the balloon 10, which may coincide with a length of the intermediate portion 24, is expandable to a diameter D2. The diameter D1 is different from, e.g., greater than, the diameter D2. With this arrangement, the balloon 10 can be used to selectively assist in expanding and/or deforming one or more portions of the stent 12 to larger diameters than other portions of the stent 12. Preferably, the first portion 26 is positioned to coincide with one of the ends 30 of the stent 12, while the second portion 28 is positioned to coincide with an intermediate section 32 of the stent 12, the intermediate section 32 being spaced from the ends 30. In this manner, at least one of the ends 30 of the stent 12 may be flared.

Optionally, a third portion 34 of the balloon 10 may be formed with a diameter D3 that is different from, e.g., larger, than the diameter D2. D3 may approximately equal D1. Preferably, the second portion 28 is located between the first and third portions 28, 34 and, more preferably, the third portion 34 is located in proximity to the second end 22. In addition, the third portion 34 is preferably positioned to coincide with one of the ends 30 of the stent 12. With locating the diameter D2 between the diameters D1 and D3, two of the ends 30 of the stent 12 may be flared.

To allow for smooth transitions between the first, second and third portions 26, 28, 34, one or more transition surfaces 36 may be provided for the balloon 10. For example, the transition surfaces 36 may be tapered or arcuate. With the arrangement of FIG. 2, the transition surfaces 36 are arcuate with the intermediate portion 24 being generally concave. End transition surfaces 38 may also be provided to connect the first and seconds 20, 22 with the first and third portions 26, 34, respectively.

The first and third portions 26, 34 may be formed generally cylindrically, as shown in FIG. 2, with varying longitudinal lengths. The spacing between the first and third portions 26, 34 should be evaluated in view of the longitudinal length of the stent 12. The spacing between the first and third portions 26, 34 may affect the extent of flaring and overall expansion of the stent 12 which is achievable. The diameters D1, D2, D3 should also be evaluated in view of the construction of the stent 12 (e.g., inherent material characteristics; permissible ratio of diameters (e.g., extent of flaring)). The profile of the balloon 10 in an expanded state will generally match the profile of the stent 12 in an expanded state.

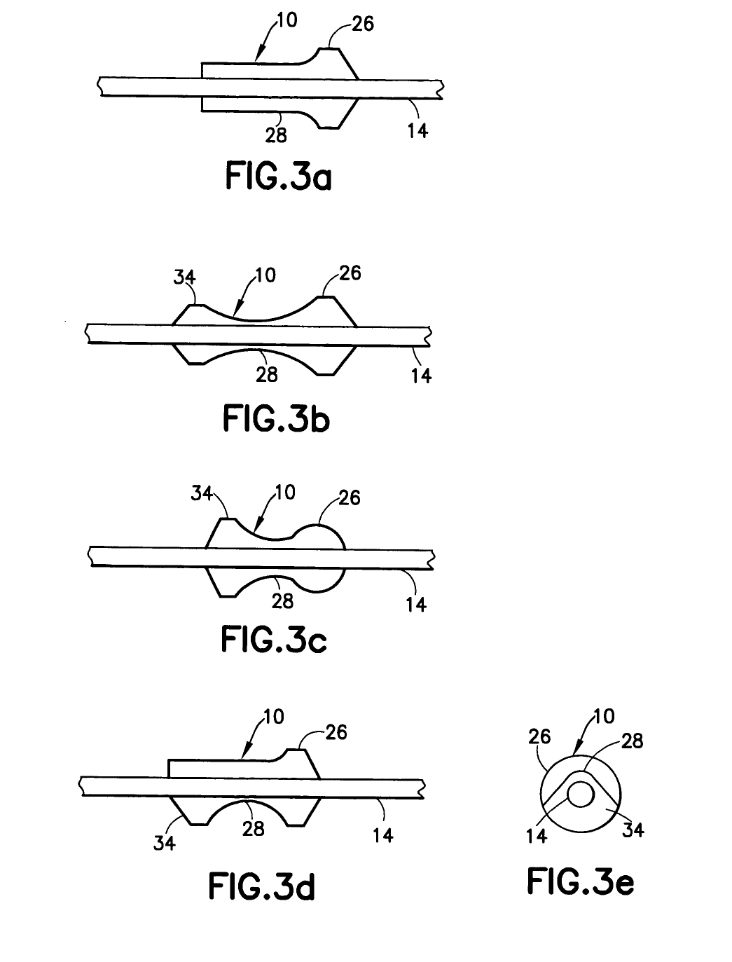

As will be appreciated by those skilled in the art, the balloon 10 may be formed with various geometries beyond that shown in FIG. 2. With reference to FIGS. 3(a)-(e), various alternative configurations of the first, second and third portions 26, 28, 34, respectively, are depicted. These configurations are by way of non-limiting examples and any geometry consistent with the principles herein may be utilized. As shown in FIG. 3(a), the third portion 34 need not be provided. Here, the first portion 26 allows for the flaring of one of the ends 30 of the stent 12. With reference to FIG. 3(b), the first and third portions 26 and 34 may be both provided, with the diameter D3 being smaller than the diameter D1. Both diameters D1 and D3, however, are greater than the diameter D2. This arrangement allows for different degrees of flaring of the ends 30 of the stent 12.

FIG. 3(c) shows that the first and third portions 26, 34 may be formed with different shapes. For example, the first portion 26 may be generally spherous, with the first diameter D1 being defined as generally the diameter of the spherous form. The third portion 34 may be generally cylindrical at the diameter D3 and extend coextensively with a portion of the catheter 14, same as in the configuration of FIG. 2.

As shown with FIGS. 3(d) and (e), the first or third portions 26, 34 may be formed to extend only partially circumferentially about the catheter 14. With the other shown configurations, the first and third portions 26, 34 extend fully circumferentially about the catheter 14. In FIGS. 3(d) and (e), the third portion 34 is shown to extend no greater than half the circumference of the catheter 14. The portion of the balloon 10 above the third portion 34 is generally coextensive with the second portion 28 (i.e., coextensive with the diameter D2). This configuration allows for a partial flaring of the ends 30 of the stent 12—the entire circumference of the ends 30 need not be flared.

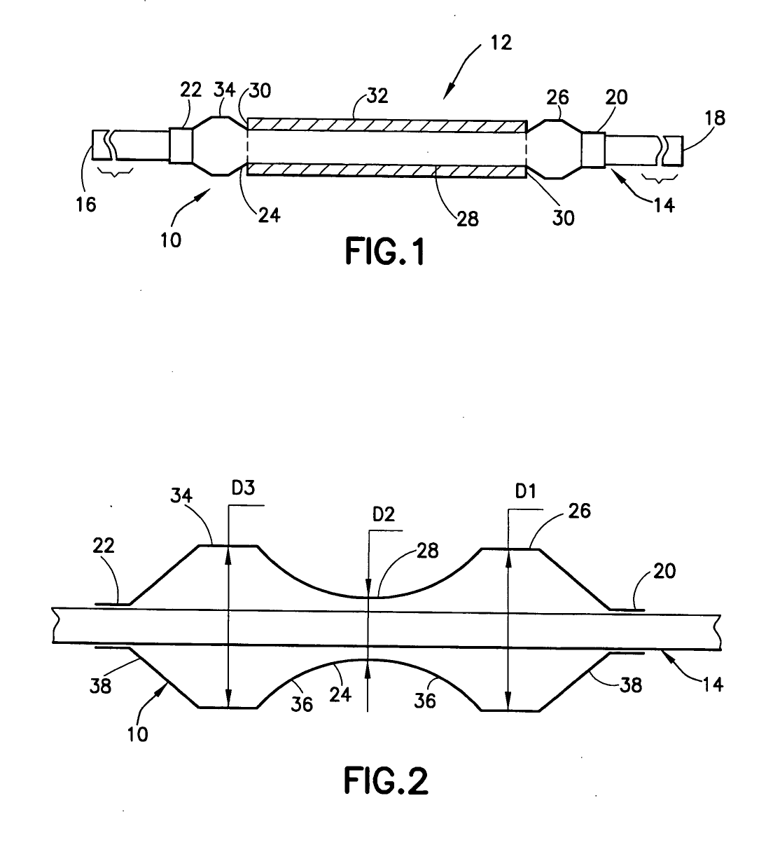

The balloon 10 may be formed of any conventional material used in balloon formation, including, but not limited to, PET, Pebax, Hytrel, nylon and combinations thereof. To allow for a non-constant profile, the balloon 10 may be initially manufactured into the desired shape. For example, the balloon 10 may be blown or molded into the finished shape, within a mold cavity resembling the final desired shape. With the balloon 10 being in the unexpanded state, excess material of the balloon 10 corresponding to the first and third portions 26, 34 collects or bunches about the catheter 14 and extends radially outwardly from the catheter 14. As shown in FIG. 1, material of the balloon 10 used in forming the first portion 26 is shown to be bunched together. Advantageously, the bunched material of the first portion 26 extends radially outwardly from the catheter 14 further than the second portion 28 and the stent 12. Consequently, the bunched material of the first portion 26 inhibits axial movement of the stent 12 along the longitudinal length of the catheter 14. With the first and third portions 26 and 34 being utilized, bunched material of the first and third portions 26, 34 in the unexpanded state may simultaneously inhibit axial movement of the stent 12 in both longitudinal directions along the length of the catheter 14.

Optionally, the balloon 10 may be formed with varying wall thickness to allow for differing extents of expansion. With thinner walls, portions of the balloon 10 (e.g., the first portion 26) may be expanded to greater diameters than portions of the balloon 10 having thicker wall portions (e.g., the second portion 28). The balloon 10 may be also formed of different materials having different resistances to expansion (e.g., different durometers). A weaker material will allow for greater expansion than a stronger, more rigid material.

The balloon 10 may be selectively expanded and deflated via the catheter 14 as required. Known techniques may be used. As discussed below, certain SMP stents require heat and, optionally, pressure for expansion. To permit heating by the balloon 10, the balloon inflation fluid may be heated, with heat from the balloon 10 being conducted to the balloon 10. Optionally, the catheter 14 may carry a heating device, e.g. a resistive heater or RF heater within the interior of the balloon 10. A heated balloon catheter is described in U.S. Pat. Nos. 5,496,311 and 4,955,377, the disclosures of which are incorporated by reference herein.

The stent 12 may be formed as a tubular structure, which may be cut or etched or otherwise have material removed or may be formed as a coiled structure resembling a coil spring. Preferably, the stent 12 is formed of SMP. As discussed in the disclosures set forth above, by way of non-limiting examples, SMP's may include polynorbornene and copolymers of polynorbornene, blends of polybornene with KRATON® (thermoplastic elastomer) and polyethylene, styrenic block copolymer elastomers (e.g., styrene-butadiene), polymethylmethacrylate (PMMA), polyethylene, polyurethane, polyisoprene, polycaprolactone and copolymers of polycaprolactone, polylactic acid (PLA) and copolymers of polyactic acid, polyglycolic acid (PGA) and copolymers of polyglycolic acid, copolymers of PLA and PGA, polyenes, nylons, polycyclooctene (PCO), polyvinyl acetate (PVAc), polyvinylidene fluoride (PVDF), blends of polyvinyl acetate/polyvinylidine fluoride (PVAc/PVDF), blends of polymethylmethacrylate/polyvinyl acetate/polyvinylidine fluoride (PVAc/PVDF/PMMA) and polyvinylchloride (PVC) and blends and/or combinations thereof.

With the stent 12 being formed of SMP, the stent 12 is pre-formed to an initial diameter. Optionally, the stent 12 may be heated near or above melt or glass transition and mechanically deformed to a smaller, contracted diameter, suitable for delivery. Alternatively, the stent 12 remains at or about its initial diameter. The stent 12 is cooled and assembled onto the catheter 14, delivered into the body of a patient, and expanded with application of heat to the melt or glass transition, while inflating the balloon 10. With the subject invention, the first portion 26 of the balloon 10 may be used to assist the expansion of a portion of the stent 12 to a diameter greater than the pre-formed initial diameter. As such, the first portion 26 may deform the stent 12 in vivo at the point of implantation. Advantageously, this allows for the stent 12 to be pre-formed without one or both of the ends 30 being initially flared, (as shown in FIG. 1), thereby allowing for a smaller overall profile for implantation. Optionally, the stent 12 may be pre-formed with some flaring at one or both of the ends 30, but with less flaring than is desired with the final configuration. In this manner, the profile of the stent 12 may be minimized, yet some shape definition may be imparted to the stent 12 to aid in formation of the flared ends.

It should also be noted that the subject invention need not deform the stent 12. Rather, the stent 12 may be pre-formed with one or both of the ends 30 being fully flared as desired. The first portion 26 and/or the third portion 34 may act to expand one or both of the ends 30 to the pre-formed flared configuration to ensure full and proper expansion into the desired pre-formed configuration, without deformation.

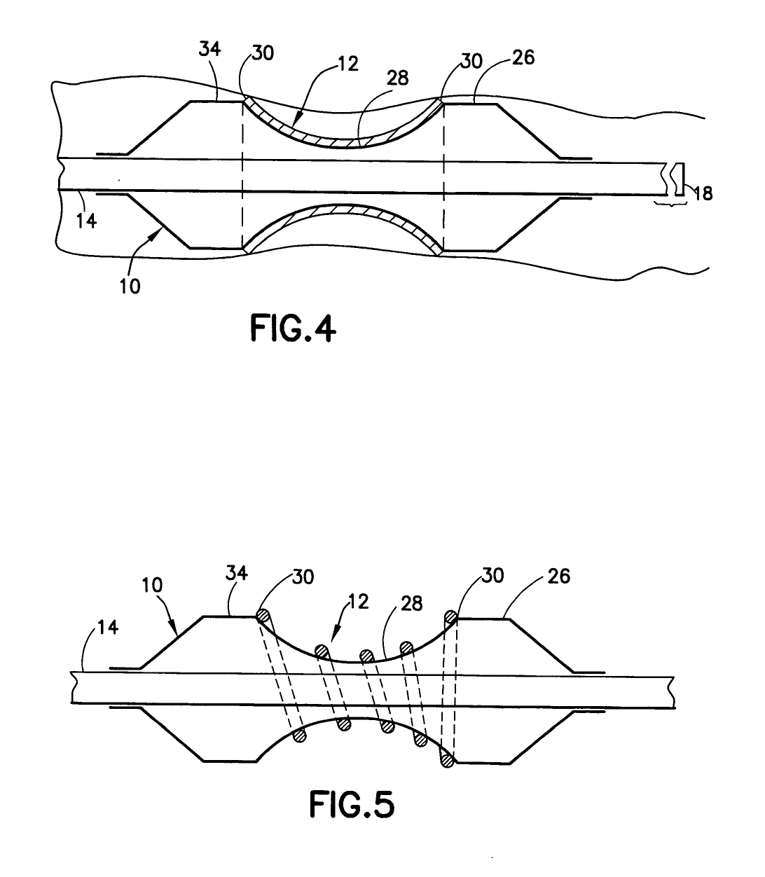

With reference to FIG. 4, the stent 12 is shown to have a tubular construction. Prior to implantation, the balloon 10 and the stent 12 are assembled, as shown in FIG. 1, with the stent 12 being mounted about the balloon 10 in an unexpanded state. The stent 12 is in a contracted or unexpanded state. A sheath or additional catheter may be placed about the assembly of FIG. 1 for implantation. To initiate implantation, the distal end 18 of the catheter 14 is inserted into the patient and guided, using known techniques, to the intended bodily passageway. The stent 12 is located within the bodily passageway at a desired location by the catheter 14 using known techniques (e.g., utilizing radiopaque markers). Thereafter, the balloon 10 is expanded, with expansion of the balloon 10 assisting flaring of one or both of the ends 30 of the stent 12. As discussed above, with the stent 12 being formed of SMP, heat is required for proper expansion. The balloon 10 and/or the catheter 14 may provide the heating as discussed above. Alternatively, the heat may be applied from a remote location outside the body. For example, as discussed in U.S. published Patent Appl. No. 2005/0010275, the SMP forming the stent 12 may be compounded to include magnetic particles, which are susceptible to heating by magnetic effects, such as hysteresis effects. A magnetic field can be imposed on the stent 12 by a source on the catheter 14 or outside the body. Heating by magnetic effects is discussed in U.S. Pat. No. 6,056,844, the disclosure of which is incorporated herein. In addition, heat may be applied by ultrasound; interfering electromagnetic beams (e.g., light beams); body heat; and/or, warm fluid through the catheter 14 (e.g., warm saline).

Once the stent 12 is expanded to its target diameter, the balloon 10 is caused to deflate as is known in the art. The catheter 14 is thereafter retracted. The flared ends 30 of the stent 12 help to anchor the stent 12 within the bodily passageway. Removal of the stent 12 can be achieved in reverse order, with heat being applied to the stent 12 to allow for its deformation to a contracted diameter.

FIG. 5 shows the stent 12 having a coiled configuration. In all basic respects, the stent 12 in the coiled configuration is expanded and implanted in the same manner as discussed above with respect to the tubular configuration.

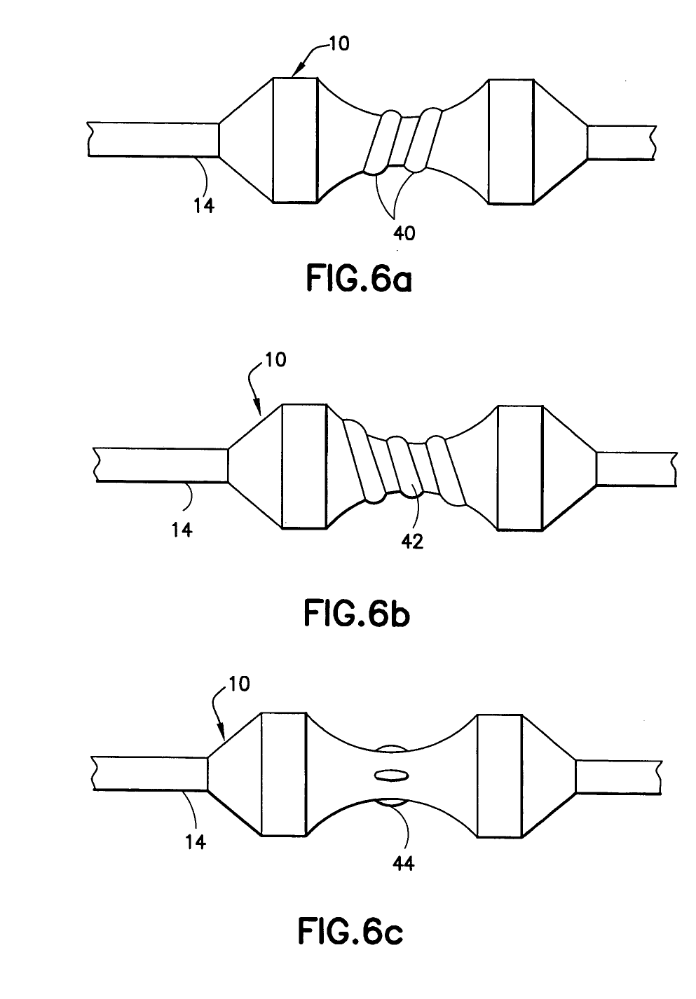

As an additional feature, the balloon 10 may be provided with one or more raised or textured features to enhance the gripping force applied to the stent 12. For example, with reference to FIGS. 6(a)-(c), the balloon 10 may be formed with: one or more rings 40 which circumscribe the circumference of the balloon 10; one or more spirals 42 which coil about the circumference of the balloon 10; and/or, one or more protrusions 44. The rings 40, spirals 42, and protrusions 44 may be unitarily formed with the balloon 10 and may be raised portions, such as defined by thickened regions of the balloon 10. The rings 40, spirals 42, and protrusions 44 are positioned to at least partially underlie the stent 12 when mounted to the balloon 10.

The stent 12 may be provided with biological and/or anti-microbial agents, as is known in the art. The stent 12 may also be provided with radiopacity.

As will be appreciated by those skilled in the art, the balloon 10 may be used with stents of various materials, including metal. For example, a metal stent (e.g., of shape memory metal (such as nitinol)) may be expanded by the balloon 10 to obtain a flared configuration. The expansion may occur below the transition temperature of the constituent metal.

As is readily apparent, numerous modifications and changes may readily occur to those skilled in the art, and hence it is not desired to limit the invention to the exact construction operation as shown and described, and accordingly, all suitable modification equivalents may be resorted to falling within the scope of the invention as claimed.

What is claimed is:

1. A combination of:
a stent for implantation in a bodily passageway; and,
a catheter having a proximal end and a distal end, a balloon being located in proximity to said distal end, said balloon being inflatable from an initial unexpanded state to an expanded state, said balloon having first and second portions, said first portion having a first diameter with said balloon being in the expanded state, said second portion having a second diameter with said balloon being in the expanded state, said first diameter being different from said second diameter, wherein said stent being mounted onto said balloon.

2. A combination as in claim 1, wherein said stent comprises a shape memory polymer.

3. A combination as in claim 2, wherein said shape memory polymer is selected from the group consisting of polynorbornene, polycaprolactone, polyenes, nylons, polycyclooctene (PCO), blends of PCO and styrene-butadiene rubber, polyvinyl acetate/polyvinylidine fluoride (PVAc/PVDF), blends of PVAc/PVDF/polymethylmethacrylate (PMMA), polymethanes, styrene-butadiene copolymers, polyethylene, trans-isoprene, blends of polycaprolactone and n-butylacrylate, POSS polyurethane polymers and blends thereof.

4. A combination as in claim 2, wherein said stent is pre-formed prior to being mounted onto said balloon, a first portion of said stent being pre-formed to a first initial diameter, wherein said first portion of said balloon assists said first portion of said stent to radially expand to a diameter at least equal to said first initial diameter.

5. A combination as in claim 4, wherein said first portion of said balloon assists said first portion of said stent to radially expand to a diameter greater than said first initial diameter.

6. A combination as in claim 4, wherein a second portion of said stent is located adjacent said second portion of said balloon, wherein said first portion of said balloon assists said first portion of said stent to radially expand more than said second portion of said stent.

7. A combination as in claim 6, wherein said stent includes first and second ends and an intermediate section therebetween, said first portion of said stent being located at said first end of said stent.

8. A combination as in claim 7, wherein said second portion of said stent being located along said intermediate section spaced from said first end of said stent.

9. A combination as in claim 4, wherein said first portion of said stent having a diameter smaller than said first initial diameter with said stent being mounted on said balloon.

10. A combination as in claim 1, wherein said first diameter is greater than said second diameter.

11. A combination as in claim 1, wherein said balloon has a third portion, said third portion having a third diameter with said balloon being in the expanded state, said third diameter being different from said second diameter.

12. A combination as in claim 11, wherein said third diameter is approximately equal to said first diameter.

13. A combination as in claim 11, wherein said second portion is located between said first portion and said third portion.

14. A combination as in claim 1, wherein said first portion is cylindrical.

15. A combination as in claim 1, wherein said first portion is spherous.

16. A combination as in claim 1, wherein, with said balloon being in the initial unexpanded state, said first portion extends radially outwardly from said second portion.

17. A combination as in claim 1, wherein said balloon includes first and second ends, said second portion of said balloon being spaced from said first and second ends.

18. A combination as in claim 17, wherein said first portion of said balloon being located between said first end of said balloon and said second portion of said balloon.

19. A method of implanting a stent in a bodily passageway of a patient, said method comprising the steps of:
providing a catheter having a proximal end and a distal end, a balloon being located in proximity to said distal end, said balloon being inflatable from an initial unexpanded state to an expanded state, said balloon having first and second portions, said first portion having a first diameter with said balloon being in the expanded state, said second portion having a second diameter with said balloon being in the expanded state, said first diameter being different from said second diameter;
mounting a stent about said balloon with said balloon being in the initial unexpanded state;
inserting said catheter into the bodily passageway of the patient;
locating said stent at a desired implantation site in the bodily passageway of the patient; and,
causing expansion of said balloon to the expanded state with said first portion of said balloon assisting a first portion of said stent to radially expand more than a second portion of said stent located adjacent said second portion of said balloon.

20. A method as in claim 19, wherein said stent is formed of a shape memory polymer.

21. A method as in claim 20, wherein said shape memory polymer is selected from the group consisting of polynorbornene, polycaprolactone, polyenes, nylons, polycyclooctene (PCO), blends of PCO and styrene-butadiene rubber polyvinyl acetate/polyvinylidine fluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate, POSS polyurethane polymers and blends thereof.

22. A method as in claim 19 further comprising, prior to the step of providing, the step of pre-forming said stent with said first portion of said stent having a first initial diameter.

23. A method as in claim 22, wherein the step of mounting includes contracting said stent with said first portion of said stent having a contracted diameter smaller than said first initial diameter.

24. A method as in claim 19, wherein the step of causing expansion includes assisting said first portion of said stent to radially expand to a diameter at least equal to said first initial diameter.

25. A method as in claim 24, wherein the step of causing expansion includes assisting said first portion of said stent to radially expand to a diameter greater than said first initial diameter.

26. A method as in claim 19, wherein said bodily passageway is selected from the group consisting of a gastrointestinal tract, esophagus, bronchi, trachea, urine tract and blood vessel.

27. A method as in claim 19, wherein said bodily passageway is a bile duct.

28. A method as in claim 19, wherein said bodily passageway is a urinary tract.

29. A combination of:
a stent for implantation in a bodily passageway; and,
a catheter having a proximal end and a distal end, a balloon being located in proximity to said distal end, said balloon being inflatable from an initial unexpanded state to an expanded state, said balloon having first and second portions, wherein, with said balloon being in the initial unexpanded state, said stent is mounted onto said balloon about said second portion, said first portion of said balloon extending radially outwardly from said catheter further than said second portion of said balloon, said first portion limiting axial movement of said stent relative to said second portion of said balloon.

30. A combination as in claim 29, wherein said balloon includes a third portion, said second portion being located between said first and third portions, wherein, with said balloon being in the initial unexpanded state, said third portion extending radially outwardly from said catheter further than said second portion of said balloon, said third portion limiting axial movement of said stent relative to said second portion of said balloon.
